(19) United States
(12) Patent Application Publication
(10) Pub. No.: US 2004/0225166 A1
(43) Pub. Date: Nov. 11, 2004

(54) Method for producing 1,1,1,3-tetrachloropropane and other haloalkanes with iron catalyst

(75) Inventors: Richard L. Wilson, Mulvane, KS (US); John L. Dawkins, Derby, KS (US)

Correspondence Address: ARMSTRONG, KRATZ, QUINTOS, HANSON & BROOKS, LLP, Suite 220, 502 Washington Avenue, Towson, MD, 21204, US

(73) Assignee: VULCAN CHEMICALS A BUSINESS GROUP OF VULCAN MATERIALS COMPANY

(21) Appl. No.: 10/648,709

(22) Filed: Aug. 26, 2003

Related U.S. Application Data
(60) Provisional application No. 60/468,008, filed on May 5, 2003.

(51) Int. Cl. C07C 17/266

(52) U.S. Cl. 570/171

(57) ABSTRACT

A continuous process is provided for the manufacture of haloalkanes by the reaction of carbon tetrachloride with olefins in the presence of iron metal, trialkylphosphate, and ferric chloride. A fraction of the catalyst and co-catalyst are separated after the reaction and recycled. In a preferred application, the olefin is ethene, and the haloalkane product is 1,1,1,3-tetrachloropropane. Two distillation steps take place in order to enhance the production of 1,1,1,3-tetrachloropropane.

### RELATED APPLICATIONS

This application is related to provisional application Ser. No. 60/468,008 filed May 5, 2003.

### FIELD OF THE INVENTION

The herein disclosed invention finds applicability in the field of haloalkane production.

### BACKGROUND OF THE INVENTION

1,1,1,3-Tetrachloropropane (HCC250fb) is useful as a feedstock to make 1-chloro-3,3,3-trifluoropropane (HCFC-253fb), which has been touted as a cleaning solvent that does not contribute substantially to ozone depletion and global warming. 1,1,1,3-Tetrachloropropane is also useful as a feedstock to produce 3,3,3-trifluoropropene (HFC-1243zf), which in turn is useful for the production of silicones and agricultural chemicals. A useful function would be served if efficient and economical methods for producing 1,1,1,3-tetrachloropropane were developed.

### DESCRIPTION OF THE PRIOR ART

Woodard describes in EP131561 a general method for the production of 1,1,1,3-tetrachloropropane by the reaction of ethene with carbon tetrachloride in the presence of iron metal and a phosphorous (V) compound. This document also mentions a variety of other alkenes and alkynes as possible alternate reactants to ethene. The patent states that the method is “adapted for continuous operation, for example in a cascaded stirred tank reaction system”, but the patent fails to provide any further details how such a continuous system might be designed. The object of the present invention is to provide an improved process for the continuous manufacture of 1,1,1,3-tetrachloropropane by the reaction of ethene and carbon tetrachloride in the presence of iron and an organophosphate co-catalyst.

Dow Corning (Great Britain 971,324) teaches the method of preparing 1,1,1,3-tetrachloropropane by reacting carbon tetrachloride with ethylene. Ditertiary butyl peroxide is a necessary ingredient in the process. Dow Corning does not disclose the process of this invention.

Thompson (U.S. Pat. No. 2,658,930) teaches a method of producing polychloroalkanes by reacting a mono-olefinic hydrocarbon and a tetrahalide in the presence of iron, water and a gas containing uncombined oxygen. The reaction is carried out at 0° C. to about 100° C. for a sufficient length of time. The preferable temperature for carrying out the process is from 25° to about 75° C. After the reaction takes place, the desired tetrahaloalkane and small amounts of hexahaloethane are removed and the unreacted mono-olefin and tetrahalide are returned to the reaction vessel. It is important to note that both water and oxygen are critical components in the Thompson process.

Asscher et al (U.S. Pat. No. 3,651,019) teaches a process directed to producing adducts of halogenated organic compounds using olefinically unsaturated substances and to certain adducts thus formed. For example, carbon tetrachloride is reacted with an olefin to produce an haloalkane product. Among the products produced were 1,1,1,3-tetrachloropropane and 1,1,1,5-tetrachloropentane. Asscher et al, however, do not teach the operative conditions of the herein disclosed invention to economically and efficiently produce the desired haloalkane product.

Takamizawa et al (U.S. Pat. No. 4,243,607) teaches the process for producing tetrachloroalkanes in which alkyl phosphites are used as part of the catalyst mixture. The herein claimed process is not disclosed.

Rygas (U.S. Pat. No. 6,187,978) teaches methods for preparing haloalkanes comprising reacting haloalkane and haloalkene starting materials in the presence of an appropriate catalyst system. Nitriles can be used in the catalyst system. Chlorinated alkenes rather than simple alkenes are used in the Rygas process.

Wilson et al (U.S. Pat. No. 6,313,360) teaches a method for preparing 1,1,1,3,3-pentachloropropane using carbon tetrachloride, vinyl chloride and iron as a catalyst. The herein disclosed invention is distinct from that of Wilson et al (U.S. Pat. No. 6,313,360) in that different starting materials are employed, different process step and reaction conditions appertain, and distinct chemical entities are produced.

None of the prior art patents disclose the inventive process herein disclosed and claimed.

### OBJECTS OF THE INVENTION

A main object of the invention is to produce chloroalkane compounds in an economical and efficient manner.

More specifically, a further object of the invention is to produce 1,1,1,3-chloropropane in an economical and efficient manner.

These and other objects of the present invention will become apparent from a reading of the following specification taken in conjunction with the enclosed drawings.

### BRIEF SUMMARY OF THE INVENTION

The invention provides a process for the manufacture of haloalkanes, in which carbon tetrachloride (CTC) and an olefin, such as ethene, propene, butene, hexene, heptene, octene, vinyl chloride, 1,1-dichloroethene, 2-chloropropene, 2-chlorobutene, etc., are reacted in the presence of a catalyst mixture comprising metallic iron, dissolved iron(II) and iron(III) components, and an organophosphate co-catalyst under conditions effective to produce an adduct of carbon tetrachloride and the olefin, and then the product mixture is distilled to produce an overhead fraction enriched in the adduct, and a bottom fraction comprising most of the catalyst components and high-boiling byproducts of the reaction, and part of the bottom fraction is recycled to the reactor.

### DESCRIPTION OF THE INVENTION

In a preferred mode, the invention provides a continuous process for the production of 1,1,1,3-tetrachloropropane, in which carbon tetrachloride and ethene are reacted in the presence of a catalyst mixture comprising metallic iron, dissolved iron(I) and iron(III) components, and an organophosphate co-catalyst under conditions effective to produce 1,1,1,3-tetrachloropropane, and then the product mixture is distilled to produce an overhead fraction enriched in 1,1,1,3-tetrachloropropane and a bottom fraction containing most of the catalyst components and high-boiling byproducts of the reaction, and then part of the bottom fraction is recycled to the reactor.

### DESCRIPTION OF THE DRAWINGS

FIG. 1 is a flow-diagram showing method steps for preparing 1,1,1,3-tetrachloropropane of the invention.

FIG. 2 is a flow-diagram showing alternative method steps for preparing 1,1,1,3-tetrachloropropane of the invention.

Reaction Step.

In a preferred embodiment, ethene, carbon tetrachloride (CTC), tributylphosphate (TBP), and recycle catalyst mixture are continually fed into a reactor. Ethene and carbon tetrachloride may be fed as liquids and/or as gasses. The two reactants may be fed separately into the reactor, or together as a mixture. TBP and recycle catalyst mixture are liquids under normal conditions, and may be fed separately or combined with other feed components. Metallic iron may be fed into the reactor either continuously, or as needed, at intervals of time. Metallic iron in any form may be used, but powder is preferred. The iron may be fed to the reactor by any feasible means, but powder slurry in a liquid feed stream (e.g. in CTC) is preferred. The reactor is an agitated vessel made of materials that are sufficiently resistant to corrosion by the reaction mixture under prevailing conditions. Nickel alloys, PTFE, tantalum, and glass-lined steel are preferred process wetted materials. The reactor is agitated for three main purposes: a) to provide adequate contact of the liquid reactants with the surface of the metallic iron, b) to provide adequate contact of the liquid with the vapor in the reactor headspace so that ethene is readily dissolved in the liquid, and c) to provide adequate contact of the reaction mixture with heat-transfer surfaces, to enable adequate temperature control. The agitation system is to be designed to accomplish all of these functions effectively.

Metallic iron is slowly consumed in the reactor, producing ferrous chloride or other dissolved iron(II) species. Ferrous chloride is slowly oxidized to ferric chloride, or to other iron(III) species. The reaction works best when all three iron oxidation states are present in the reactor. This is accomplished by including both recycle catalyst and metallic iron in the reactor feeds, and by regulating the feed rates and the reactor temperature correctly. While the dissolved iron species have herein been called ferrous chloride and ferric chloride, it is recognized that the dissolved iron may be complexed to a greater or lesser extent with organophosphate co-catalyst or with other components of the reaction mixture, including traces of water.

The feeds to the reactor will contain traces of water, varying from about 1 to 5000 ppm of water. It is preferred that the reaction mixture made from the combined feeds be substantially dry, containing from about 5 to 1000 ppm of water. Lower concentrations are feasible, but are perhaps more costly to maintain. Higher concentrations, up to a level that results in a separate aqueous phase, are also feasible, but are less desired because of increased corrosion in certain kinds of metallic equipment.

TBP is also consumed slowly in the reactor, producing chlorobutane and organophosphate byproducts. The organophosphate byproducts of degradation are believed to be less effective co-catalysts for the desired reactions than is TBP. Reactor operating conditions are chosen to produce a high yield of the desired 1,1,1,3-tetrachloropropane product, high ethene conversion, low selectivity to 1,1,1,3-tetrachloropentane and higher boiling byproducts, and low degradation of the organophosphate co-catalyst. The following table lists the preferred reactor operating conditions.

| Condition | Units | Range | Preferred Range |
|---|---|---|---|
| reactor temperature | C | 80 to 140 | 90 to 125 |
| reactor pressure | psig | 10 to 300 | 40 to 200 |
| feed ratios | | | |
| CTC/ethene | mole/mole | 1.0 to 3.0 | 1.1 to 2.0 |
| TBP/metallic iron | mole/mole | 1.0 to 2.0 | 1.01 to 1.3 |
| metallic iron/ethene | mole/mole | 0.001 to 0.006 | 0.002 to 0.006 |
| dissolved iron/ethene | mole/mole | 0.01 to 0.10 | 0.02 to 0.08 |

The dissolved iron/ethene feed ratio is controlled by regulating the flow rate of the recycle catalyst, which contains all or most of the dissolved iron fed to the reactor.

The reactor effluent stream is preferably treated, using a solid-liquid separation device, to separate coarse iron particles from the liquid, and to return the coarse iron particles to the reactor. The relatively coarse iron particles contain most of the iron mass present in the reactor, and therefore should be kept in that location. The device should allow particulate fines (e.g. insoluble impurity inclusions from the iron powder, and relatively small iron particles) to pass into the subsequent distillation stage, whence they can be eventually purged from the system. The solid-liquid separation device can be a centrifuge, a hydrocyclone, a filter, a sedimentation tube, etc. A sedimentation tube provides a relatively quiet zone, separating coarse particles from reactor effluent and facilitating the return of coarse iron to the reaction mixture.

Catalyst Recovery Step.

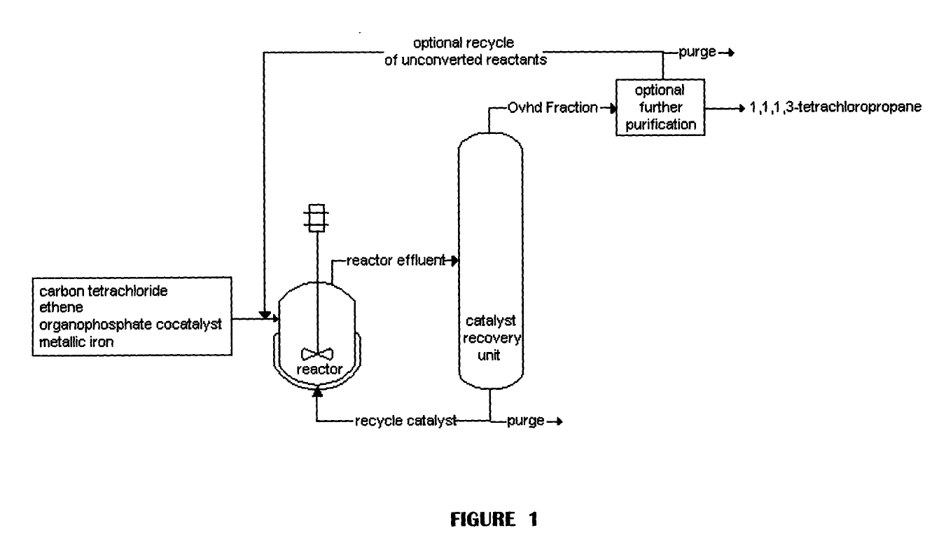

In a preferred embodiment, the reactor effluent passes into a catalyst recovery unit, which separates the reactor effluent into fractions by distillation. It produces an overhead fraction containing most of the 1,1,1,3-tetrachloropropane product and a bottom fraction containing most of the catalyst components. The overhead fraction also contains any unconverted reactants ethene and CTC. These may be separated from the desired product, 1,1,1,3-tetrachloropropane, in subsequent purification steps and returned to the reactor or purged from the system. The bottom fraction contains some of the 1,1,1,3-tetrachloropropane produced in the reactor, in addition to the various dissolved iron species and phosphorus-containing species. Although we suppose that the iron species are primarily metallic iron, iron(II) chloride and iron(III) chloride, this is not known with certainty. The phosphorus-containing species include organophosphate co-catalyst, and products that result from the degradation of the organophosphate co-catalyst. The balance of the bottoms fraction is primarily comprised of 1,1,1,5-tetrachloropentane and 1,1,1,7-tetrachloroheptane, and higher molecular weight compounds of similar nature, and degradation products thereof. As one preferred alternative (FIG. 1), this bottoms fraction may be returned to the reactor as recycle catalyst without further treatment. Some of the bottoms fraction may be purged to rid the system of excessive degradation products.

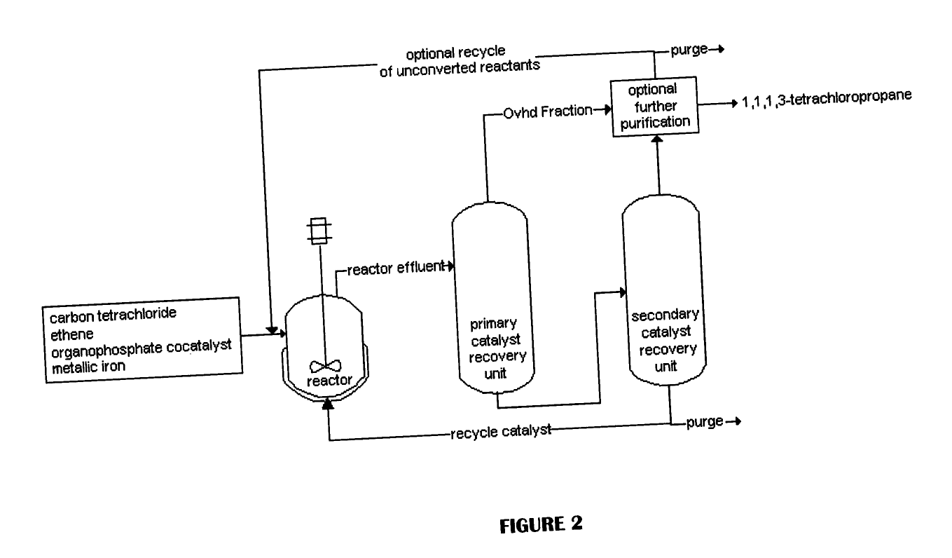

In another preferred alternative (FIG. 2), the bottom fraction from the primary catalyst recovery unit may be distilled again, in a secondary catalyst recovery unit, under different conditions, to produce an overhead fraction containing an increased concentration of the desired product 1,1,1,3-tetrachloropropane, and a new bottoms fraction containing an increased concentration of the catalyst components. The latter may be recycled to the reactor as recycle catalyst, or partly purged. The overhead fraction from this second distillation step may, if it is pure enough, be combined with the rest of the purified 1,1,1,3-tetrachloropropane product, or it may be further purified.

The temperature in the catalyst recovery unit, and in the secondary catalyst recovery unit, if there is one, is regulated to prevent excessive degradation of the catalyst components and of the 1,1,1,3-tetrachloropropane product. The pressure of these units is regulated to achieve the desired operating temperature. Preferably, the temperature in these units is below 115° C., and more preferably, it is below 105° C. Degradation is increased not only by increasing temperature but also by increasing liquid residence time. Therefore, the liquid residence time in these units is preferably minimized. It is possible, and it may be appropriate, to trade decreased liquid residence time for increased temperature. The reason for this is that decreased temperature requires decreased pressure, which may be costly.

This is related to one reason that the process of FIG. 2 may turn out to be preferable to that of FIG. 1. The primary catalyst recovery unit needs to be fairly large, because it takes the entire reactor effluent as feed. This means that the liquid residence time in this unit will be long. Therefore, to keep degradation processes under control, the temperature must be kept low, which implies either very low overhead pressure (costly) or relatively low concentration of the catalyst components in the boiling liquid (also costly, unless there is a secondary recovery unit). The catalyst components all have extremely low vapor pressures. The secondary catalyst recovery unit can be quite small, which permits higher operating temperatures and pressures, while still keeping degradation processes under control. Preferably, the overhead distillation pressure in these units is between 5 and 850 torr, and more preferably between 40 and 225 torr.

The “optional further purification” shown in these diagrams will generally consist of distillation steps and perhaps a drying step. These may be designed according to principles known in the prior art.

### DETAILED DESCRIPTION OF THE INVENTION

A method for preparing a chloroalkane compound comprising reacting ethene with carbon tetrachloride in the presence of an iron catalyst and a tributylphosphate co-catalyst. The reaction taking place over a period of time of up to several days, at a temperature of up to about 140° C. and a pressure of up to about 200 psig. Once the reaction takes place, solids are separated and the reaction mixture is distilled to produce an overhead fraction rich in 1,1,1,3-tetrachloropropane and a bottom fraction containing catalyst components and high-boiling byproducts, with the bottom fraction being recycled to the reactor. The overhead fraction besides containing 1,1,1,3-tetrachloropropane, also, may contain un-reacted ethene and CTC which are separated from the 1,1,1,3-tetrachloropropane with the ethene and CTC being returned to the reactor. The bottoms fraction contains some 1,1,1,3-tetrachloropropane, iron and phosphorous co-catalyst. Also contained in the bottoms fraction are other byproducts. The bottoms fraction may be recycled to the reactor or the bottoms fraction may be distilled again in a secondary catalyst recovery unit (CRU) to produce an overhead fraction of 1,1,1,3 tetrachloropropane and a bottoms fraction of concentrated catalyst components. The temperature in the secondary CRU is such as to slow degradation of the catalysts as well as the 1,1,1,3-tetrachloropropane product. The liquid residence time is less than 12 hours, and preferably less than six hours, in order to restrict degradation of components. Since the primary catalyst recovery unit needs to be fairly large, the liquid residence time of less than five days, and preferably less than one day will be a longer residence time than required for the smaller secondary CRU. To keep degradation under control, the temperature must be kept low at 70 to 115° C. Its liquid inventory is on the order of 1-5% of the inventory in the reactor, or even smaller.

A method is provided for preparing a chloroalkane compound comprising reacting ethene with carbon tetrachloride, in the presence of a metallic iron and ferric chloride catalyst and a tributylphosphate co-catalyst. The preferred molar feed ratios are from 1.0 to 3.0 moles carbon tetrachloride per mole ethene, from 0.02 to 0.10 mole ferric chloride per mole ethene, from 1.01 to 1.20 mole tributylphosphate per mole ferric chloride. The reaction is performed in the presence of an amount of iron metal, which may be granulated, powdered, or in any other convenient shape or form, the amount being adjusted to provide the desired rate of reaction. If the iron is in the form of a smaller than 100-mesh powder, for example, the amount will ordinarily be about 0.001 to 0.010 moles per mole of ethene feed. The temperature of the reaction will preferably be between 80° and 140° C., and more preferably between 90° and 130° C. The pressure of the reaction will be adjusted to attain the desired rate of reaction, and will preferably lie between 30 and 200 psig. If the reaction is performed as a batch reaction, then the reaction time will be chosen to attain the desired degree of ethene conversion; preferably the batch time will be between 0.2 and 20 hours. The desired degree of ethene conversion will generally lie between 50 and 100%, and more preferably between 60 and 98%. If the reaction is performed as a continuous reaction, the residence time shall be chosen to attain the desired degree of conversion; preferably the residence time will lie between 0.2 and 48 hours where the residence time is calculated as the volume of the reactor divided by the volumetric flow of liquid reactor effluent. More preferably, the residence time will be between 1 and 20 hours.

Once the reaction is complete, to the desired level of conversion, the reaction mixture is separated from solids (mainly unconsumed metallic iron) and then distilled at a bottom temperature of from 70 to 115° C., and at pressure of 40 to 225 torr overhead pressure, to produce an overhead fraction rich in 1,1,1,3-tetrachloropropane and a bottom fraction containing catalyst components, 1,1,1,3-tetrachloropropane, and unwanted high-boiling byproducts. The liquid residence time in this distillation tower is less than five days, and preferably less than one day, where the liquid residence time is calculated as the tower liquid inventory divided by the volumetric flow rate of liquid from the tower bottom. A fraction of the bottom fraction is removed from the system to control the concentration of the unwanted byproducts, and another fraction is recycled to the reactor to provide the needed ferric chloride feed, and much of the needed tributylphosphate feed. The overhead fraction, besides containing 1,1,1,3-tetrachloropropane also may contain unreacted ethene and CTC which are separated from the 1,1,1,3-tetrachloropropane by distillation or other conventional means. A fraction of the recovered ethene and CTC are returned to the reactor.

In an alternate implementation, which is optimized to recover a greater fraction of the 1,1,1,3-tetrachloropropane from the reactor effluent without degrading the catalyst components too much, the bottom fraction from the distillation described above may be distilled a second time to recover more 1,1,1,3-tetrachloropropane product from it. In this two-stage catalyst recovery implementation, the first stage (as described above) recovers in the distillate between 50 and 90% of the 1,1,1,3-tetrachloropropane contained in the reactor effluent, leaving more than 98% of the high-boiling unwanted byproducts in the bottom. Then the second stage recovers in the distillate more than 70% of the remaining 1,1,1,3-tetrachloropropane contained in the bottoms fraction from the first stage. This second distillation is performed at a bottom temperature of from 70 to 115° C., and at from 40 to 225 torr overhead pressure, with the liquid residence time in the tower bottom being short, preferably less than 12 hours, and more preferably less than 6 hours, the liquid residence time being calculated as the volume of liquid in the tower bottom divided by the volumetric flow rate of liquid from the tower bottom. The overhead from this second distillation, being mainly 1,1,1,3-tetrachloropropane, may be further purified as desired. A fraction of the bottom liquid is recycled to the reactor to provide the needed ferric chloride feed, and much of the needed tributylphosphate feed. Another fraction, preferably between 5 and 20 percent, may be removed from the system to control the concentration of unwanted byproducts in the system.

Said another way the invention involves a method for producing a haloalkane comprising reacting ethene, carbon tetrachloride, tributylphosphate, and iron in a reactor to produce a reaction effluent, separating the reaction effluent by distillation into an overhead fraction of desired haloalkane, ethene and carbon tetrachloride and a bottom fraction of catalyst components for return to the reactor. In the distillation step of the effluent, the overhead contains 1,1,1,3-tetrachloropropane as well as ethene and carbon tetrachloride. Further, the ethene and carbon tetrachloride can be separated by distillation and returned to the reactor. The reactor effluent, which contains 1,1,1,3-tetrachloropropane, 1,1,1,5-tetrachloropentane and higher molecular weight compounds, is subjected to distillation at a temperature of 70 to 115° C. and at a pressure of 40 to 225 torr to obtain the purified 1,1,1,3-tetrachloropropane. Preferably the distillation temperature is 80 to 100° C. and the pressure is 62 to 134 torr, and most preferably the distillation temperature is 90° C. and the pressure is 194 torr. In the process of this invention, the bottoms fraction can be returned to the reactor after removal of the 1,1,1,3-tetrachloropropane.

In an alternative embodiment of this invention, the bottom fraction is subjected to a second distillation at a temperature of 70-115° C. after the first distillation of at 70-115° C. in order to produce an increased concentration of 1,1,1,3-tetrachloropropane and the residue of the distillation containing catalyst components may be recycled to the reactor.

In the inventive process, the reaction in the reactor is carried out at under 150° C. or below 130; with the reaction time in the reactor being between 0.2 and 20 hours. Exemplary conditions for the reactor are a temperature of 90-130° C. and the pressure of 30-200 psig.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A process for preparing haloalkanes which comprises:
a) mixing and heating carbon tetrachloride with an olefin in a reactor in the presence of metallic iron, dissolved iron species, and an organophosphate co-catalyst to produce a continuous flow of reactor effluent containing a haloalkane product,
b) treating the reactor effluent from step a) with a solid-liquid separation device that separates coarse solid iron particles from the liquid and returns the course solid iron particles to the reactor, to produce by this separation a reactor effluent containing a decreased concentration of coarse solid iron particles,
c) distilling the reactor effluent from step b) in a catalyst recovery unit to produce a continuous flow of overhead product containing an increased concentration of product haloalkane and a continuous flow of bottom product containing increased concentrations of dissolved iron and phosphorus-containing species that are active in the catalysis of the reaction to make product haloalkane, and
d) recycling a portion of the bottom product from step c) to the reactor while purging the rest from the system.

2. The process of claim 1 wherein the organophosphate co-catalyst is trialkylphosphate.

3. The process of claim 2 wherein the trialkylphosphate co-catalyst is tributylphosphate or tripropylphosphate or triisobutylphosphate.

4. The process of claim 1 wherein the main inputs to the reactor are: carbon tetrachloride, ethene, tributylphosphate, metallic iron, and recycle catalyst from the catalyst recovery unit, and the product haloalkane is 1,1,1,3-tetrachloropropane.

5. The process of claim 1 wherein the solid-liquid separation device is a centrifuge, a hydrocyclone, a filter or a sedimentation tube.

6. A process for preparing haloalkane which comprises:
a) mixing and heating carbon tetrachloride with an olefin in a reactor in the presence of metallic iron, dissolved iron species, and an organophosphate co-catalyst to produce a continuous flow of reactor effluent containing product haloalkane, and
b) treating the reactor effluent from step a) with a solid-liquid separation device that separates coarse solid iron particles from the liquid and returns them to the reactor, to produce a reactor effluent containing a decreased concentration of coarse particles, and
c) distilling the reactor effluent from step b) in a catalyst recovery unit to produce a continuous flow of overhead product containing an increased concentration of product haloalkane and a continuous flow of bottom product containing increased concentrations of dissolved iron and phosphorus-containing species which are active in the catalysis of the reaction to make product haloalkane, and
d) distilling the bottom product from step c) in a continuous or batch secondary catalyst recovery unit to produce an overhead product containing an increased concentration of product haloalkane and a bottom product containing a increased concentrations of dissolved iron and phosphorus-containing components that are active in the catalysis of the reaction to make product haloalkane, and
e) recycling a portion of the bottom product from step d) to the reactor while purging the rest from the system.

7. The process of claim 6 wherein the organophosphate co-catalyst is trialkylphosphate.

8. The process of claim 7 wherein the trialkylphosphate co-catalyst is tributylphosphate or tripropylphosphate or triisobutylphosphate.

9. The processes of claim 6 wherein the main inputs to the reactor are: carbon tetrachloride, ethene, tributylphosphate, metallic iron, and recycle catalyst from the catalyst recovery unit, and the product haloalkane is 1,1,1,3-tetrachloropropane.

10. The process of claim 6 wherein the solid-liquid separation device is a centrifuge, a hydrocyclone, a filter, and a sedimentation tube.

11. A method of producing a haloalkane comprising reacting ethene, carbon tetrachloride, tributylphosphate, and iron in a reactor to produce a reaction effluent, separating the reaction effluent by distillation into an overhead fraction of desired haloalkane, ethene and carbon tetrachloride and a bottom fraction of catalyst components for return to the reactor, in the distillation step of the effluent, the overhead containing 1,1,1,3-tetrachloropropane as well as ethene and carbon tetrachloride.

12. In the process of claim 11 wherein the ethene and carbon tetrachloride are separated by distillation and returned to the reactor.

13. In the process of claim 11 wherein the bottoms fraction, which contains 1,1,1,3-tetrachloropropane, 1,1,1,5-tetrachloropentane and higher molecular weight compounds, is subjected to distillation at a temperature of 70 to 115° C. and at a pressure of 40 to 225 torr to obtain the purified 1,1,1,3-tetrachloropropane.

14. The process of claim 13 wherein the distillation temperature is 80 to 105° C. and the pressure is 62 to 160 torr.

15. The process of claim 14 wherein the distillation temperature is 93° C. and the pressure is 103 torr.

16. The process of claim 13 wherein the bottoms fraction is returned to the reactor after removal of the 1,1,1,3-tetrachloropropane.

17. The process of claim 11 wherein the bottom fraction is subjected to a second distillation at a temperature of 70-115° C. after the first distillation at 70-115° C. in order to produce an increased concentration of 1,1,1,3-tetrachloropropane and the residue of the distillation containing catalyst components may be recycled to the reactor.

18. The process of claim 11 wherein the reaction in the reactor is carried out at below 150° C.

19. The process of claim 18 wherein the process is carried out at below 130° C.

20. The process of claim 19 wherein the reaction time in the reactor is between 0.2 and 20 hours.

21. In the process of claim 11, the liquid residence time is 0.2 to 20 hours; the temperature is 90-130° C. and the pressure is 30-200 psig.

22. A method for preparing 1,1,1,3-tetrachloropropane comprising the steps of
a) reacting ethene with carbon tetrachloride, in the presence of a metallic iron and ferric chloride catalyst and a tributylphosphate co-catalyst.
b) separating the solids from the liquid reaction mixture,
c) distilling the liquid reaction mixture at a temperature of from 70 to 115° C., and at pressure of from 40 to 225 torr overhead pressure, to produce an overhead fraction rich in 1,1,1,3-tetrachloropropane and a bottom fraction containing catalyst components, 1,1,1,3-tetrachloropropane, and unwanted high-boiling byproducts.
d) distilling the bottom fraction a second time at a temperature of 70 to 115° C. and at 40 to 225 torr to obtain additional 1,1,1,3-tetrachloropropane.
